(19) United States
(12) Patent Application Publication
(10) Pub. No.: US 2017/0215898 A1
(43) Pub. Date: Aug. 3, 2017

(54) DEVICE FOR REMOVING A THROMBUS FROM A RETINAL VESSEL

(71) Applicant: Tetsu ASAMI, Nagoya-shi (JP)

(72) Inventor: Tetsu ASAMI, Nagoya-shi (JP)

(21) Appl. No.: 15/376,088

(22) Filed: Dec. 12, 2016

(30) Foreign Application Priority Data
Feb. 3, 2016 (JP) 2016-018614

(51) Int. Cl. A61B 17/22; A61F 9/007; A61B 34/00

(52) U.S. Cl. CPC A61B 17/22; A61B 34/72; A61F 9/007; A61B 2017/00345

(57) ABSTRACT

A device for removing a thrombus from a retinal vessel has a grip held by the user of the device, a tubular portion to be inserted into the eye, the tubular portion being provided at an end of the grip, a microtube to be inserted into a retinal vessel, the microtube being provided at an end of the tubular portion, a linear pusher axially slidably held in the microtube and the tubular portion, and an operating portion provided at the grip. The linear pusher is adapted to protrude to a desired position from an end of the microtube by axially sliding the linear pusher by means of the operating portion, thereby the linear pusher physically pushes a thrombus out of the retinal vessel.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to a device for removing a thrombus from a retinal vessel. The device for removing a thrombus from a retinal vessel according to the present invention is used mainly in a treatment for branch retinal vein occlusion. The device for removing a thrombus from a retinal vessel according to the present invention would be usable also for treatments of central retinal vein occlusion, central retinal artery occlusion, branch retinal artery occlusion, etc.

2. Description of Related Art Including Information Disclosed Under 37 CFR 1.97 and 37 CFR 1.98.

Retinal vessel occlusion is classified into retinal artery occlusion in which a retinal artery is occluded and retinal vein occlusion in which a retinal vein is occluded. Retinal artery occlusion is classified into central retinal artery occlusion in which a central retinal artery within optic nerves is occluded and the whole retina falls into ischemia, and branch retinal artery occlusion in which a branch retinal artery is occluded and part of the retina falls into ischemia. Retinal vein occlusion is classified into central retinal vein occlusion in which a central retinal vein within optic nerves is occluded and causes bleeding, and branch retinal vein occlusion in which a branch retinal vein is occluded and causes bleeding. Conventional treatments for retinal artery occlusion include eye massage, administering a vasodilator or a thrombolytic agent, and anterior chamber tap. Conventional treatments for branch retinal vein occlusion include dripping tissue plasminogen activator (tPA) for the purpose of dissolving a vascular thrombosis and discission of a retinal vessel sheath (sheathotomy) in vitreous surgery for the purpose of releasing vein compression by an artery at a retinal arteriovenous crossing site. A recent treatment for central retinal vein occlusion comprises a micro needle having a width of 50 μm being inserted into a central retinal vein, tPA being injected into said central retinal vein to dissolve a thrombus.

In the conventional treatments in which tPA, which is a thrombus dissolving agent, is dripped or directly injected into a central retinal vein by means of a micro needle in the expectation that the thrombus will be dissolved by enzyme, but it is not clear to what extent the thrombus is actually dissolved.

In said discission of retinal vessel sheath at a retinal arteriovenous crossing site, vein compression by an artery is released but the thrombus within the vein is not removed.

In said conventional treatments for retinal artery occlusion comprising eye massage, administering a vasodilator or a thrombolytic agent, and anterior chamber tap, it is possible to slightly move the thrombus within the vessel, but it is difficult to remove the thrombus.

### BRIEF SUMMARY OF THE INVENTION

It is an object of the invention to provide a device for removing a thrombus from a retinal vessel, said device eliminating all of said disadvantages of said conventional treatments.

It is another object of the invention to provide a device for removing a thrombus from a retinal vessel, which can remove a thrombus from a vessel by physically pushing the thrombus out of the retinal vessel.

These and other objects have been attained by the following device for removing a thrombus from a retinal vessel according to the present invention.

(1) A device for removing a thrombus from a retinal vessel comprising:
a grip held by the user of the device;
a tubular portion to be inserted into the eye, said tubular portion being provided at an end of said grip;
a microtube to be inserted into a retinal vessel, said microtube being provided at an end of said tubular portion;
a linear pusher axially slidably held in said microtube and said tubular portion;
an operating portion provided at said grip;
said linear pusher being adapted to protrude to a desired position from an end of said microtube by axially sliding said linear pusher by means of said operating portion, thereby said linear pusher physically pushing a thrombus out of said retinal vessel.

(2) Said operating portion comprises:
a longitudinal opening formed in said grip;
a slide control portion being disposed in said longitudinal opening;
said linear pusher being slid longitudinally by sliding said slide control portion longitudinally in said longitudinal opening.

(3) Said microtube is curved.

(4) Said linear pusher is fixed in a reinforcing member, said reinforcing member being connected with said operating portion.

(5) Said linear pusher is elastically curved according to the curve of the retinal vessel.

(6) Said linear pusher is made of nitinol.

According to the present invention, a thrombus is physically pushed out of the retinal vessel. Therefore, the thrombus is surely removed from a retinal vessel.

### BRIEF DESCRIPTION OF THE SEVERAL VIEWS OF THE DRAWINGS

FIG. 1 is a plan view showing an example of a device for removing a thrombus from a retinal vessel according to the present invention.

FIG. 2 is a side view showing said example of said device according to the present invention.

FIG. 3 is a sectional view showing said example of said device according to the present invention.

FIG. 4 is a sectional view showing an end portion of said device according to the present invention.

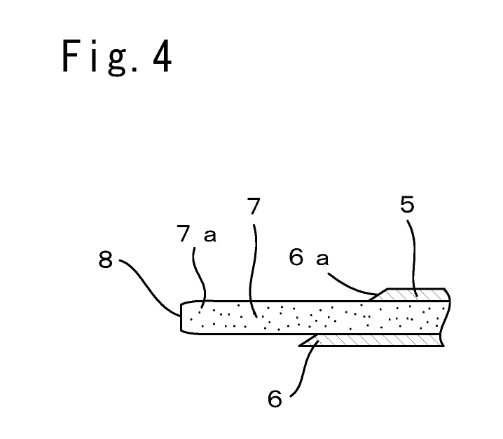

FIG. 5 is a plan view showing a microtube of said device according to the present invention.

FIG. 6 is an enlarged longitudinal sectional view showing a portion of said device according to the present invention.

FIG. 7 is an enlarged cross-sectional view showing a portion of said device according to the present invention.

FIG. 8 is an explanatory view showing an example of said device according to the present invention used in vitreous surgery.

FIG. 9 is an explanatory view showing said device inserted into a retinal vessel to remove a thrombus from said retinal vessel.

### DETAILED DESCRIPTION OF THE INVENTION

The present invention will now be described in detail with reference to the attached drawings.

A device 1 for removing a thrombus from a retinal vessel according to the present invention comprises, a grip 2 held by the user of the device 1, a tubular portion 3 to be inserted into the eye, said tubular portion 3 being provided at an end of said grip 2, a microtube 5 to be inserted into a retinal vessel 21, said microtube 5 being provided at an end of said tubular portion 3, a linear pusher 7 axially slidably held in said microtube 5 and said tubular portion 3, an operating portion 18 provided at said grip 2, said linear pusher 7 being adapted to protrude to a desired position from an end of said microtube 5 by axially sliding said linear pusher 7 by means of said operating portion 18, thereby said linear pusher 7 physically pushing a thrombus 22 out of said retinal vessel 21.

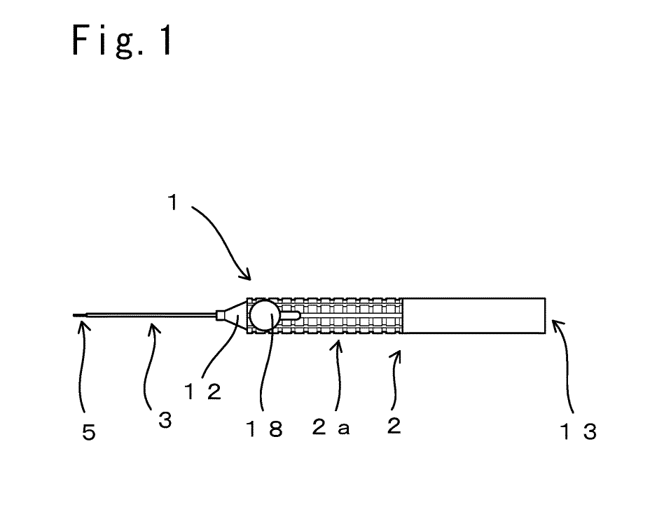

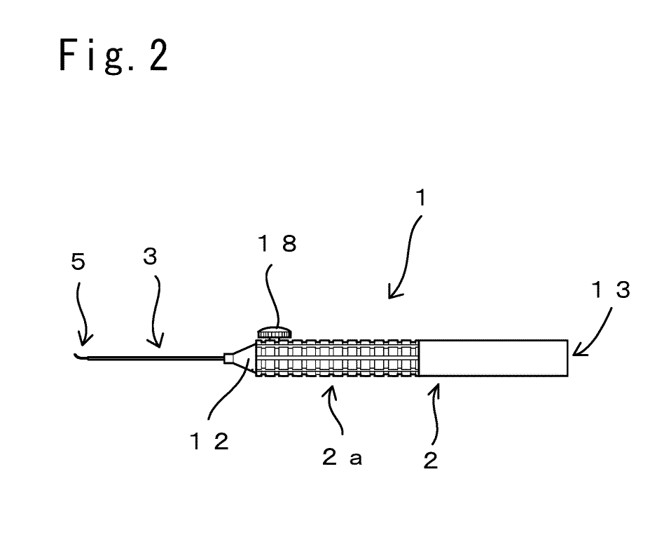

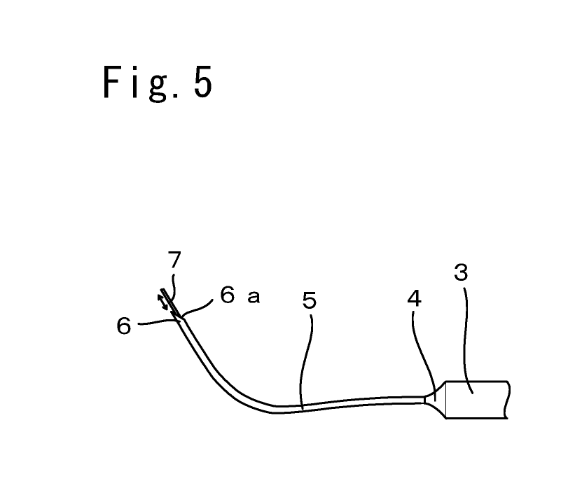

Said tubular portion 3 is inserted into the eye in vitreous surgery. Said tubular portion 3 is preferably straight as shown in FIGS. 1 and 2. Said tubular portion 3 may be curved. Said linear pusher 7 is slidably inserted into said tubular portion 3. Said tubular portion 3 is preferably made of a metal. Said tubular portion 3 preferably has about 27 gauge (diameter of 0.36 mm). Said tubular portion 3 preferably has a length of about 31 mm. An end 4 of said tubular portion 3 may have a shape of a frustum as shown in FIG. 5.

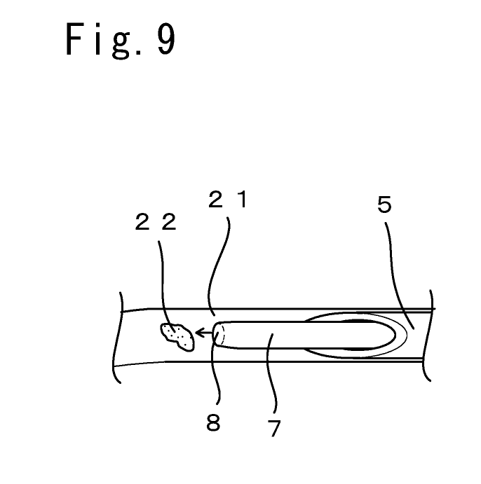

Said microtube 5 is inserted into a retinal vessel 21. See FIG. 9. Said microtube 5 is preferably curved as shown in FIG. 5. A curved microtube 5 is easier to insert into the retinal vessel 21. Alternatively the microtube 5 may be straight. The microtube 5 is provided at an end 6 thereof with a cutting face 6a preferably on an inside of the curve of said microtube 5 as shown in FIG. 5, that is, on an upper side of said end of the microtube 5 in FIG. 5 so that the cutting face 6a can be easily seen by the user of the device 1. The microtube 5 is curved preferably at an angle of about 30 to 90 degrees, more preferably at an angle of about 60 degrees, with respect to the axis of the device 1, according to the position, etc. of the retinal vessel 21. The angle of the curve of the microtube 5 may be adjusted minutely by means of a needle holder, etc. The cutting face 6a at the end 6 of the microtube 5 may be inclined at any angle with respect to the axis of the microtube 5 as long as the microtube 5 is easily inserted into the retinal vessel 21. A preferable angle of inclination of the cutting face 6a at the end 6 of the microtube 5 may be between about 30 and 45 degrees with respect to the axis of the microtube 5. The linear pusher 7 is slidably inserted into the microtube 5. The microtube 5 preferably has an outer diameter of about 0.08 mm and a length of about 5 mm.

Said grip 2 is held with one hand by the user of the device 1 in vitreous surgery. The grip 2 is made of a synthetic resin or a metal. The grip 2 preferably has a cylindrical shape. A front end 12 of the grip 2 may have a shape of a frustum as shown in FIG. 1. The grip 2 may have an anti-slip means 2a on the periphery thereof as shown in FIG. 1 so that said grip 2 does not slip in the hand of the user.

Said linear pusher 7 is inserted into the retinal vessel 21 and physically pushes a thrombus 22 out of said retinal vessel 21 as shown in FIG. 9. The linear pusher 7 is preferably a solid cylindrical body having a roundish end 8. Thereby, the linear pusher 7 inserted into the retinal vessel 21 can remove the thrombus 22 without hurting the endothelium of the retinal vessel 21. The linear pusher 7 is preferably made of a material having superelasticity and shape-memory capability. Said material withstands curving and bending, and when deformed returns to its predeformed shape. Said material must be biocompatible. Said material may be nitinol, which has been used in medical devices. Materials other than nitinol may be used if they have elasticity or superelasticity, shape-memory capability and biocompatibility similar to those of nitinol. Materials such as certain synthetic resins and alloys may be used.

The device 1 according to the present invention may be used in a curved retinal vessel 21. Superelastic materials such as said nitinol are flexible enough to be elastically curved according to the curve of the vessel. When the device 1 according to the present invention is used in a treatment for central retinal vein occlusion, said microtube 5 is inserted into a branch retinal vein and said linear pusher 7 is moved toward a central retinal vein. On the way thereto, the vein has a large curve at an optic nerve head. When the device 1 according to the present invention is used in a treatment for central retinal artery occlusion, said microtube 5 is inserted into a branch retinal artery and said linear pusher 7 is moved toward a central retinal artery. On the way thereto, the artery has a large curve at an optic nerve head. When the device 1 according to the present invention is used in a treatment for central retinal vein occlusion or said central retinal artery occlusion, it is necessary to curve said linear pusher 7 at an angle of about 90 degree downward, that is, toward the inner part of the eye at the curved portion. Therefore, when the device 1 according to the present invention is used in a treatment for central retinal vein occlusion or said central retinal artery occlusion, it is necessary to use a linear pusher 7 made of a flexible material. To prevent the endothelium of the retinal vessel at said curved portion from being hurt, an end portion 7a of said linear pusher 7 may be made of a material more flexible than other portions or the end portion 7a of said linear portion 7 may be covered with a flexible, roundish cap.

Said linear pusher 7 is connected with said operating portion 18. The linear pusher 7 is protruded from the end 6 of said microtube 5 and retracted into said microtube 5 by operating the operating portion 18.

The maximum length of protrusion of said linear pusher 7 from said end 6 of the microtube 5 is probably about 3 to 5 mm, but not limited thereto because the preferable length of protrusion of said linear pusher 7 from said end 6 of the microtube 5 varies according to the portion of the vessel into which the linear pusher 7 is inserted. The diameter of the linear pusher 7 is preferably about 0.06 mm, but not limited thereto because the preferable diameter varies according to the portion of the vessel into which the linear pusher 7 is inserted.

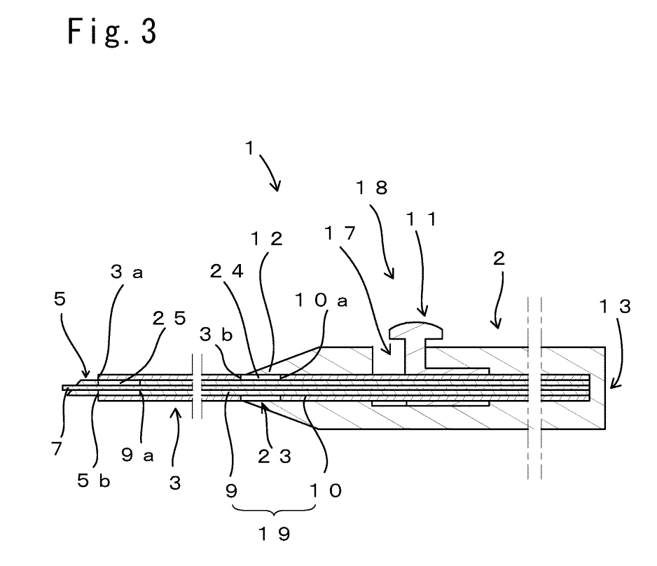

In FIG. 3, said linear pusher 7 extends to a position near a rear end 13 of the device. However, the linear pusher 7 does not necessarily extend to a position near said rear end 13 of the device. The linear pusher 7 may have any length provided that the length of protrusion of the linear pusher 7 can be adjusted by means of the operating portion 18. The linear pusher 7, at least a portion thereof protrusible from the end 6 of the microtube 5, is preferably made of said material such as nitinol. The overall length of the linear pusher 7 is not necessarily made of the same material. A portion of the linear pusher 7 not protrusible from the end 6 of the microtube 5 may be made of a material other than said material such as nitinol.

Said operating portion 18 comprises a longitudinal opening 17 formed in said grip 2, and a slide control portion 11 disposed in said longitudinal opening 17. Said linear pusher 7 is slid longitudinally by sliding said slide control portion 11 longitudinally in said longitudinal opening 17.

In vitreous surgery, the user holds the grip 2 with one hand. Said linear pusher 7 is preferably slid by a simple operation.

Said operating portion 18 including said slide control portion 11 may be directly connected with said linear pusher 7. For the reinforcement of the linear pusher 7, the linear pusher 7 is preferably fixed in a reinforcing member 19, said reinforcing member 19 being connected with said operating portion 18. The linear pusher 7 moves together with the reinforcing member 19.

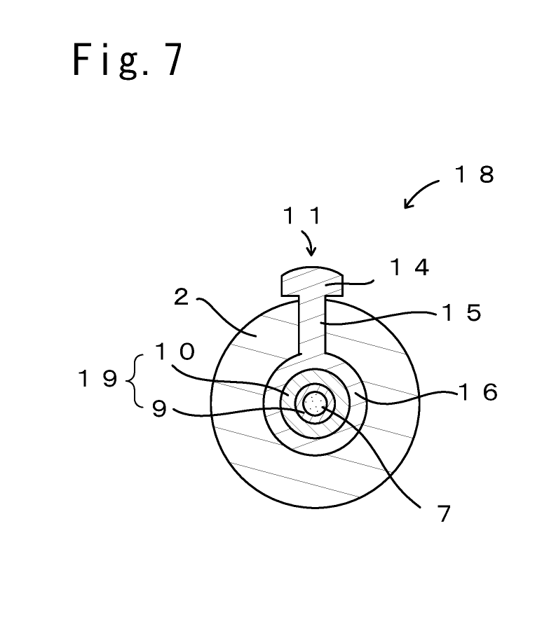

The reinforcing member 19 is preferably a tube. Said linear pusher 7 is inserted into said tube and fixed thereto so as to move together with said tube. The reinforcing member 19 moves forward and backward together with the linear pusher 7 in a central hole 23 formed longitudinally within said grip 2. In an example shown in FIGS. 3, 6 and 7, said reinforcing member 19 comprises a first tube 9 directly fixed around said linear pusher 7 and a second tube 10 fixed around said first tube 9.

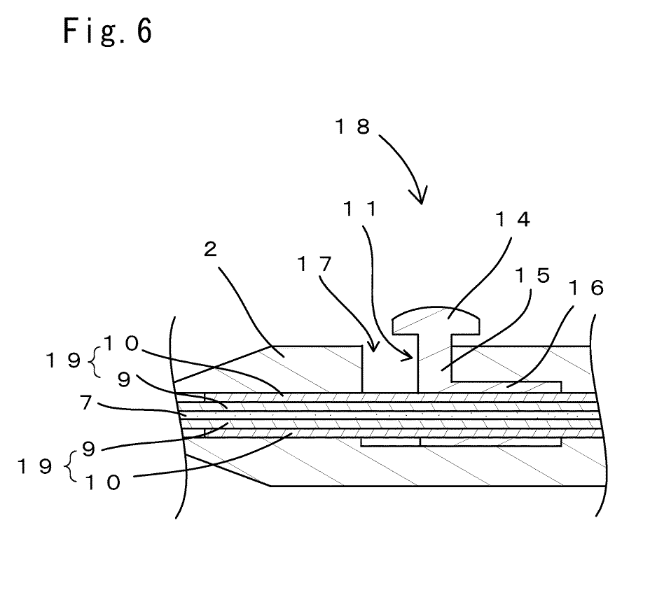

Said slide control portion 11 is slidable with respect to said grip 2. The slide control portion 11 protrudes from said grip 2. The slide control portion 11 is fixed to said linear pusher 7 or to said reinforcing member 19. The slide control portion 11 comprises, for example, an annular portion 16 fixed around said linear pusher 7 or said reinforcing member 19, a fingertip operating portion 14 protruding from the grip 2 so as to be easily operated by fingertips, and a connecting portion 15 connecting said annular portion 16 with said fingertip operating portion 14. Said connecting portion 15 is located within said longitudinal opening 17 formed in said grip 2. See FIG. 6.

In an example shown in FIG. 3, a first space 24 is formed within said central hole 23 between a front end 10a of said second tube 10 in said reinforcing member 19 and a rear end 3b of said tubular portion 3 fixed to said front end 12 of said grip 2, a second space 25 being formed within said tubular portion 3 between a front end 9a of said first tube 9 in said reinforcing member 19 and a rear end 5b of said microtube 5 fixed to a front end 3a of said tubular portion 3. When said second tube 10 within said central hole 23 moves in a direction toward or away from said rear end 3b of said tubular portion 3, said first tube 9 moves in a direction toward or away from said rear end 5b of said microtube 5 so as to protrude said linear pusher 7 from said microtube 5 or move said linear pusher 7 back into said microtube 5. The maximum length of said first space 24, the maximum length of said second space 25 and the maximum distance of movement of said fingertip operating portion 14 within said longitudinal opening 17 are approximately the same. Said linear pusher 7 moves forward or backward within the range of said maximum length or said maximum distance.

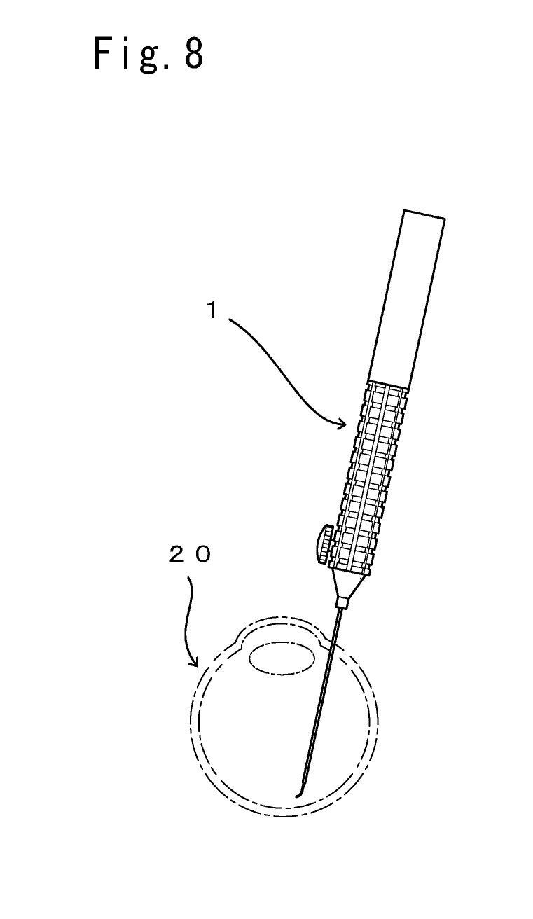

FIG. 8 shows an example of the device 1 according to the present invention used in vitreous surgery. The user holds the grip 2, inserts the tubular portion 3 into the eye 20, inserts the microtube 5 into a retinal vessel 21, protrudes the linear pusher 7 into the retinal vessel 21, and removes a thrombus 22 from the retinal vessel 21.

As many apparently widely different embodiments of the present invention may be made without departing from the spirit and scope thereof, it is to be understood that the invention is not limited to the specific embodiments thereof except as defined in the appended claims.

I claim:

1. A device for removing a thrombus from a retinal vessel comprising:
a grip held by the user of the device;
a tubular portion to be inserted into the eye, said tubular portion being provided at an end of said grip;
a microtube to be inserted into a retinal vessel, said microtube being provided at an end of said tubular portion;
a linear pusher axially slidably held in said microtube and said tubular portion;
an operating portion provided at said grip;
said linear pusher being adapted to protrude to a desired position from an end of said microtube by axially sliding said linear pusher by means of said operating portion, thereby said linear pusher physically pushing a thrombus out of said retinal vessel.

2. The device of claim 1, wherein said operating portion comprises:
a longitudinal opening formed in said grip;
a slide control portion being disposed in said longitudinal opening;
said linear pusher being slid longitudinally by sliding said slide control portion longitudinally in said longitudinal opening.

3. The device of claim 1, wherein said microtube is curved.

4. The device of claim 1, wherein said linear pusher is fixed in a reinforcing member, said reinforcing member being connected with said operating portion.

5. The device of claim 1, wherein said linear pusher is elastically curved according to the curve of the retinal vessel.

6. The device of claim 1, wherein said linear pusher is made of nitinol.
